(19) United States
(12) Patent Application Publication
(10) Pub. No.: US 2006/0157062 A1
(43) Pub. Date: Jul. 20, 2006

(54) Mouth guard retention apparatus, athletic uniform and method

(76) Inventor: Paul Raymond, Boscawen, NH (US)

Correspondence Address: Michael J. Persson;Lawson & Persson, P.C., Suite 103, 67 Water Street, Laconia, NH, 03246, US

(21) Appl. No.: 11/371,498

(22) Filed: Mar. 9, 2006

Related U.S. Application Data
(63) Continuation of application No. 10/458,959, filed on Jun. 11, 2003, now abandoned.
(60) Provisional application No. 60/388,207, filed on Jun. 13, 2002.

(51) Int. Cl. A61G 15/00; A61F 5/37

(52) U.S. Cl. 128/845; 128/846; 128/848; 128/859; 128/860; 128/861; 128/862

(57) ABSTRACT

An apparatus for preventing the loss or contamination of mouth guards, and an athletic uniform incorporating such an apparatus. The apparatus includes a strip of hook and loop fastener dimensioned to wrap around and secure the mouth guard, and a means for attaching the strip of hook and loop fastener to an athlete's uniform. The athletic uniform includes an article of clothing to which the apparatus is attached.

### CLAIM OF PRIORITY

This application is a continuation of U.S. patent application Ser. No. 10/458,959 filed on Jun. 11, 2003, which claimed the benefit of priority of U.S. Provisional Patent Application Ser. No. 60/388,207, filed on Jun. 13, 2002.

### FIELD OF THE INVENTION

The present invention relates to the field of athletic mouth guards and, in particular, to a retention apparatus, and athletic uniform including the same, for preventing the loss, contamination, or inadvertent switching of a mouth guard when it is removed from an athlete's mouth.

### BACKGROUND OF THE INVENTION

Athletes in many sports use mouth guards to protect against the loss of teeth. In fact, most intramural, interscholastic, and collegiate sports programs require the use of such mouth guards by all participants. In these programs, if an athlete does not have a mouth guard, they are not allowed to participate.

One problem common to the use of mouth guards is the loss or contamination of guards when they are removed from the user's mouth. Because most uniforms do not include pockets, or other means for retaining the guards with the person, the guards are typically placed down on benches or the ground, resulting in frequent loss or contamination of the guards. In addition, as mouth guards are all similar, and are not amenable to marking with an athlete's name, placing mouth guards down on benches or the ground creates a likelihood that the mouth guard of one athlete will be inadvertently switched with the mouth guard of another athlete; with the attendant spread of germs.

In sports in which helmets are utilized, these problems have been addressed by the inclusion of elongated ties or strap members adapted to be attached to the face guard or chin strap of the helmet. Examples of such mouth guards are shown in U.S. Pat. Nos. 2,847,003; 2,521,039; 3,312,218; 3,314,423; 3,411,501; 3,448,738 and 3,485,242. However, in sports that do not utilize helmets, there is no effective way of retaining the guards. Accordingly, coaches, athletic directors and other officials in these programs typically purchase spare guards to allow athletes to continue to participate even if their own guards are lost or contaminated.

Therefore, there is a need for an apparatus for preventing the loss, contamination, and/or switching, of mouth guards and may be used by athletes in sports in which helmets are not worn.

### SUMMARY OF THE INVENTION

The present invention is an apparatus for preventing the loss or contamination of mouth guards, and an athletic uniform incorporating such an apparatus. In its most basic form, the apparatus of the present invention includes a strip of hook and loop fastener dimensioned to wrap around and secure the mouth guard, and a means for attaching the strip of hook and loop fastener to an athlete's uniform or shoe.

In the preferred embodiment of the apparatus, the strip of hook and loop fastener includes a first surface that is entirely comprised of the hook portion of the fastener, and a second surface that is entirely comprised of a loop portion of the fastener. This arrangement is preferred as this type of fastener material is readily available and is cheaply and easily formed into the desired strip. However, in other embodiments, two pieces of material, each having one substantially smooth side and one side having hook portions and loop portions respectively, are joined together to form the strip. In some such embodiments, the substantially smooth side is emblazoned with a school or team logo, or with a manufacturer's trademark.

In the preferred embodiment of the apparatus, the means for attaching the strip of hook and loop fastener to the athlete's uniform or shoe is a pair of substantially parallel slits, which are disposed lengthwise through the center of the strip and are dimensioned to allow a string, belt, or other portion of the uniform or shoe to be disposed therethrough. In this manner, the strip is effectively attached to the outside of the uniform or shoe and may be utilized to secure the mouth guard thereto. In other embodiments of the apparatus, the means for attaching the strip to the uniform or shoe is a second strip of hook and loop fastener that is attached to the uniform or shoe via thread, adhesive, or other art recognized means, and is dimensioned to contact and secure the first strip of hook and loop fastener to the uniform or shoe. In still other embodiments, the means for attaching the strip is a snap that is dimensioned to attach to a mating snap on the uniform or shoe for easy removal and attachment of the strip.

In its most basic form, the uniform or shoe of the present invention includes an article of clothing and a strip of hook and loop fastener disposed upon the article of clothing and dimensioned to wrap around and secure the mouth guard.

Therefore, it is an aspect of the invention to provide an apparatus for preventing the loss or contamination of mouth guards that may be used by athletes in sports in which helmets are not worn.

It is a further aspect of the invention to provide an apparatus for preventing the loss or contamination of mouth guard that is relatively easy and cheap to manufacture.

It is a further aspect of the invention to provide an apparatus for preventing the loss or contamination of mouth guard that may be adapted to display a school or team logo or a manufacturer's trademark.

It is a further aspect of the invention to provide an apparatus for preventing the loss or contamination of mouth guard that may be integrated an article of clothing to form a uniform.

These aspects of the invention are not meant to be exclusive and other features, aspects, and advantages of the present invention will be readily apparent to those of ordinary skill in the art when read in conjunction with the following description and accompanying drawings.

### BRIEF DESCRIPTION OF THE DRAWINGS

FIG. 1 is a front view of the preferred apparatus of the present invention.

FIG. 2 is a side view of apparatus of FIG. 1.

FIG. 3 is a side isometric view of one embodiment of the apparatus securing a mouth guard to an article of clothing.

FIG. 4 is a front view of an alternative embodiment of the apparatus of the present invention.

FIG. 5 is a side view of the embodiment of FIG. 4.

FIG. 6 is a front view of the embodiment of FIG. 4 showing the ends overlapped in engaged relation and one smooth side emblazoned with a logo.

FIG. 7 is a front view of another alternative embodiment of the apparatus of the present invention.

FIG. 8 is a side view of the embodiment of FIG. 7.

### DETAILED DESCRIPTION OF THE INVENTION

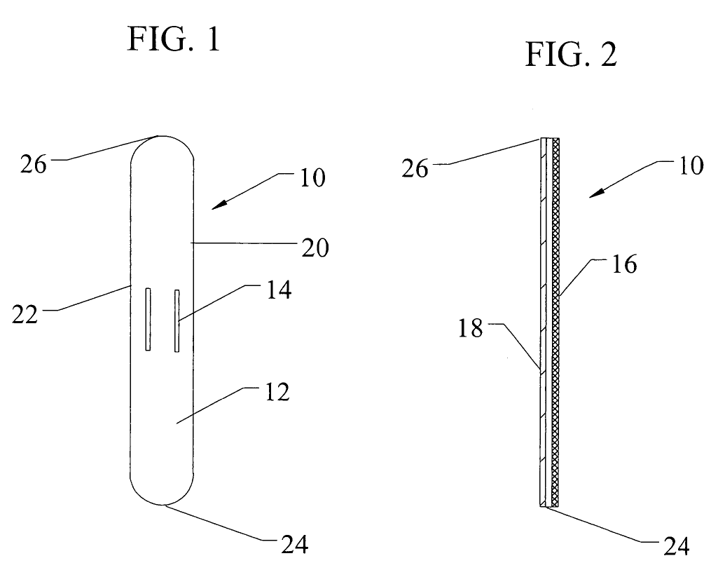

Referring first to FIGS. 1 and 2, the preferred embodiment of the apparatus 10 is shown. The apparatus of the present invention includes a strip 12 of hook and loop fastener dimensioned to wrap around and secure the mouth guard (not shown), and a means for attaching the strip of hook and loop fastener to an athlete's uniform or athletic shoes (not shown). In the preferred embodiment, the means for attaching the strip of hook and loop fastener to the uniform or shoes is a pair of substantially parallel slits 14. As shown in FIG. 1, these slits 14 are disposed lengthwise through the center of the strip 12 in a direction parallel to the edges 20, 22 of the strip 12 and are dimensioned to allow a string, belt, or other portion of the uniform or shoe to be disposed therethrough. However, in other embodiments utilizing slits 14, the slits 14 may be disposed in other manners, such as perpendicular to, or at angles from, the edges 20, 22 of the strip 12. Regardless of how the slits 14 are disposed, the slits 14 are effective at attaching the strip 12 to the outside of the uniform or the laces of a shoe (not shown).

The preferred embodiment of the apparatus 10 has a first major surface 16, that is substantially covered with the hook portions of the hook and loop fastener, and a second surface 18 that is substantially covered by the loop portions of the hook and loop fastener. This arrangement is preferred as this type of fastener material is readily available and is cheaply and easily formed into the desired strip. It is noted that either surface 16, 18 may be disposed against the outside of the uniform or shoe (not shown), but that the second surface 18, containing the loop portions, is preferably in such contact in order to prevent inadvertent scratching of exposed skin by the hook portions disposed upon the first surface 16.

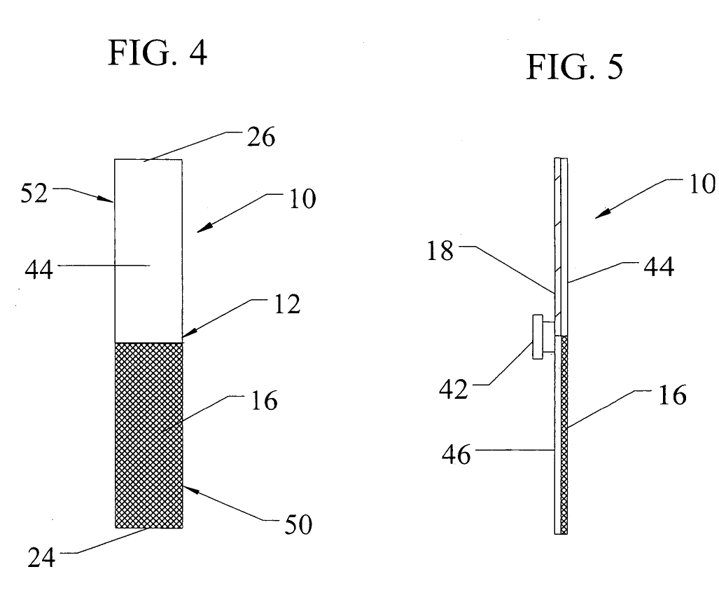

The strip 12 of the present invention may take many shapes and may be manufactured in a number of sizes. As shown in FIGS. 1 and 2, the strip is an elongate rectangle having rounded ends. However, the ends may be cut to be straight, as in FIGS. 4 and 5, triangular, or any other desired shape. Similarly, some embodiments of the strip 12 are significantly wider than those shown herein, allowing the apparatus 10 to completely or substantially surround the mouth guard.

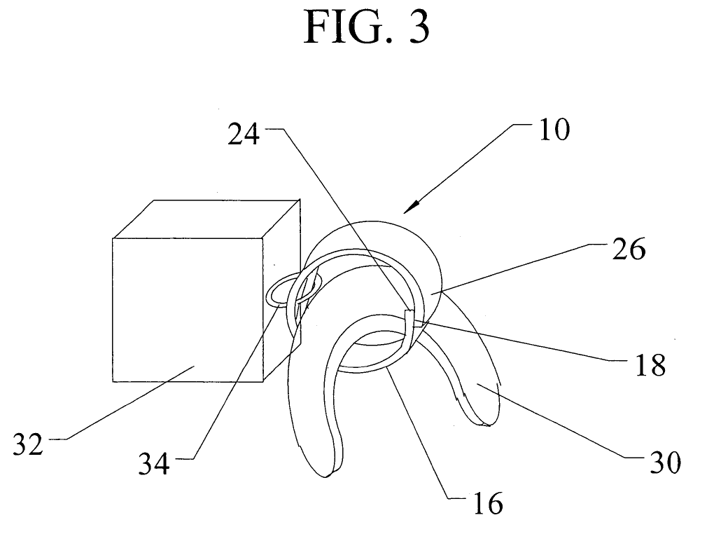

As shown in FIG. 3, the preferred apparatus 10 is attached to an article of clothing 32 by looping a string 34, which is part of, or is attached to, the article of clothing 32, through the slits 14 in the strip 12 and tying off the string 34. Once the apparatus 10 is attached to the article of clothing 32, it is ready to retain a mouth guard 30. This retention is accomplished by disposing the mouth guard 30 against the strap 12 and wrapping the ends 24, 26 of the strap 12 around the mouth guard 30 and overlapping the ends 24, 26 such that the mouth guard 30 is held in place and such that the hook portions of the fastener on the first surface 16 engage the loop portions of the fastener on the second surface 18.

Referring now to FIGS. 4 and 5, another embodiment of the apparatus 10 is shown. In this embodiment, two pieces of material 50, 52, each having one substantially smooth surfaces 44, 46 and one surface 16, 18 having hook portions and loop portions respectively, are joined together to form the strip 12. As shown in FIG. 5, the smooth surfaces 44, 46 of the two pieces of material 50, 52 are disposed in opposite directions from each other such that the hook portions disposed upon the first surface 16 will engage the loop portions disposed upon the second surface 18 when the ends 24, 26 are overlapped.

The embodiment of FIGS. 4 and 5 also utilizes a different means for attaching the strip 12 to the article of clothing (not shown). Here, the means is a portion 42 of a snap type fastener that is dimensioned to engage a mating portion (not shown) attached to the article of clothing. In this embodiment, the portion 42 is a female portion dimensioned to accept a male portion of the fastener. However, other embodiments of the apparatus 10 may utilize a male portion to achieve similar results. In still others, the means for attaching the strip to the uniform or shoe is a second strip of hook and loop fastener that is attached to the uniform or shoe via thread, adhesive, or other art recognized means, and is dimensioned to contact and secure the adjacent surface 16 or 18, of the strip 12 to the uniform or shoe.

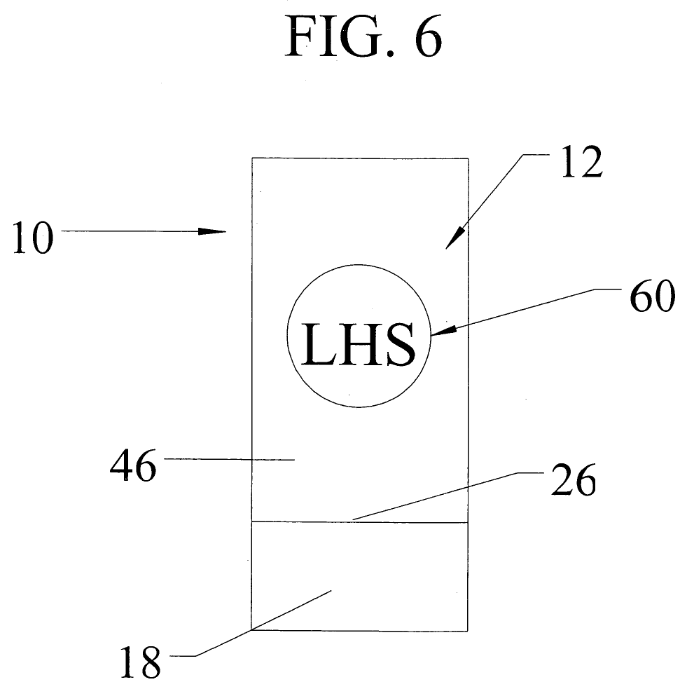

A strip 12 utilizing two separate pieces of material 50, 52 is more costly than the embodiment of the strip 12 of FIGS. 1-2 and, therefore, is not preferred. However, the smooth sides 44, 46 provided in this embodiment are advantageous in cases where the apparatus 10 is purchased by a team, or is distributed by a company as a “give away” item. In these instances, it is envisioned that the substantially smooth side will have a desired color and/or be emblazoned with a school or team logo, or with a manufacturer's trademark. One such embodiment is shown in FIG. 6. As shown in FIG. 6, a logo 60 is emblazoned upon the smooth side 46 of one of the pieces of wrapped over the other end (not shown) such that the logo 60 will be prominently displayed.

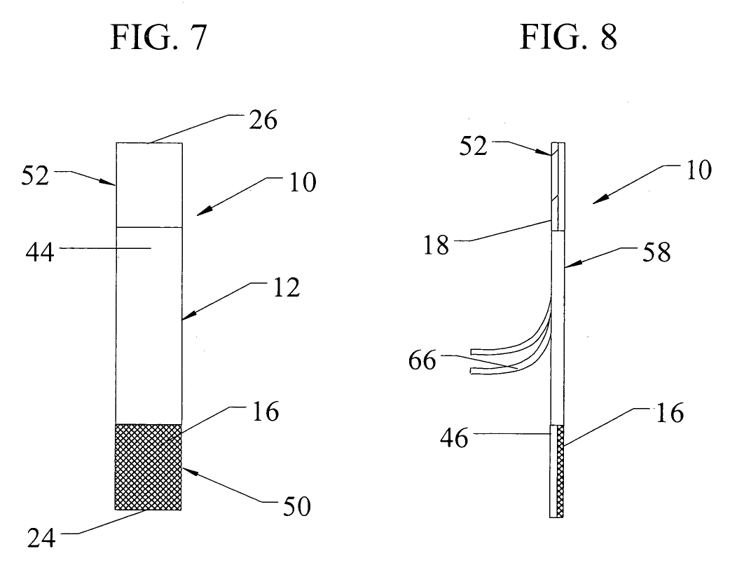

Referring now to FIGS. 7 and 8, still another embodiment of the apparatus 10 of the present invention is shown. In this embodiment, two pieces of material 50, 52, each having one substantially smooth surface 44, 46 and one surface 16, 18 having hook portions and loop portions respectively, are joined at opposite ends of a central piece of material 58 to form the strip 12. In this embodiment, the means for attaching the strip 12 to an athlete's uniform or shoe is a pair of strings 66 or laces, which allow the strip 12 to be tied onto the uniform or shoes. As shown in FIGS. 7 & 8, the strings 66 are permanently attached to the strip 12 by sewing, adhesive, or other means. However, in other such embodiments the strip 12 includes slits, such as those shown in FIGS. 1 and 2, and the pair of strings 66 is a single string that is looped through the slits and tied to the strip 12.

As noted above, it is envisioned that, in addition to the separate attachment of the apparatus described herein, the apparatus will be integrated into new athletic uniforms. Accordingly, the present invention likewise encompasses such uniforms into which the apparatus is integrated. In these embodiments, the uniform includes an article of clothing, such as a pair of shorts, tee-shirt, baseball pant, or the like, and a strip of hook and loop fastener disposed upon the article of clothing and dimensioned to wrap around and secure the mouth guard. This strip may be removably attached in the manner described above with regard to the apparatus, or it may permanently attached to the article of clothing by sewing, gluing, riveting, or by any other art recognized means for permanently attaching things to articles of clothing.

Although the present invention has been described in detail with reference to certain preferred versions thereof, other versions would be readily apparent to those of ordinary skill in the art. Therefore, the spirit and scope of the appended claims should not be limited to the description of the preferred versions contained herein.

What is claimed is:

1. An attachment apparatus for releasably securing an athletic mouth guard during periods of athletic inactivity to an athletic uniform, said apparatus comprising:
an elongate strip having a first surface comprising a hook portion of a hook and loop fastener, and a second surface comprising a loop portion of said hook and loop fastener, wherein said strip is sufficiently flexible and dimensioned to wrap about the athletic mouth guard such that said first surface of said strip attaches to said second surface of said strip to secure the athletic mouth guard within said strip; and
a means for attaching said strip to the athletic uniform.

2. The apparatus of claim 1 wherein substantially all of said first surface comprises said hook portion of said hook and loop fastener and wherein substantially all of said second surface comprises said loop portion of said hook and loop fastener.

3. The apparatus of claim 1 wherein said first surface includes a first portion comprising said hook portion of said hook and loop fastener and a substantially smooth second portion; and
wherein said second surface comprises a first portion comprising said loop portion of said hook and loop fastener and a substantially smooth second portion.

4. The apparatus of claim 3 wherein at least one of said substantially smooth first portion of said first surface and said second surface comprises at least one symbol affixed thereto.

5. The apparatus of claim 4 wherein said symbol is selected from the group consisting of a word, a number, a phrase, a picture, and a logo.

6. The apparatus of claim 1 wherein said means for attaching said strip to the athletic uniform comprises at least one interior aperture disposed through said first surface and said second surface

7. The apparatus of claim 6 wherein said interior aperture is a slit dimensioned to receive a button on the athletic uniform.

8. The apparatus of claim 6 wherein said means for attaching said strip to the athletic uniform comprises at least two interior apertures disposed through said first surface and said second surface, wherein said apertures are dimensioned and positioned to allow a shoelace to be inserted through both of said apertures.

9. The apparatus of claim 1 wherein said means for attaching said strip to the athletic uniform is a first snap portion dimensioned to attach to a mating second snap portion disposed upon the athletic uniform.

10. An athletic uniform comprising:
an article of athletic clothing; and
an attachment apparatus for releasably securing an athletic mouth guard during periods of athletic inactivity to said article of athletic clothing, said apparatus comprising;
an elongate strip having a first surface comprising a hook portion of a hook and loop fastener, and a second surface comprising a loop portion of said hook and loop fastener, wherein said strip is sufficiently flexible and dimensioned to wrap about the athletic mouth guard such that said first surface of said strip attaches to said second surface of said strip to secure the athletic mouth guard within said strip; and
a means for attaching said strip to said article of athletic clothing.

10. The athletic uniform of claim 10 wherein substantially all of said first surface of said strip comprises said hook portion of said hook and loop fastener and wherein substantially all of said second surface of said strip comprises said loop portion of said hook and loop fastener.

11. The athletic uniform of claim 10 wherein said first surface of said strip comprises a first portion comprising said hook portion of said hook and loop fastener and a substantially smooth second portion; and
wherein said second surface of said strip comprises a first portion comprising said loop portion of said hook and loop fastener and a substantially smooth second portion.

12. The athletic uniform of claim 11 wherein at least one of said substantially smooth first portion of said first surface of said strip and said second surface of said strip comprises at least one symbol affixed thereto.

13. The athletic uniform of claim 12 wherein said symbol is selected from the group consisting of a word, a number, a phrase, a picture, and a logo.

14. The athletic uniform of claim 10 wherein said means for attaching said strip to the athletic uniform comprises at least one interior aperture disposed through said first surface and said second surface

15. The athletic uniform of claim 14 wherein said interior aperture is a slit dimensioned to receive a button on the athletic uniform.

16. The athletic uniform of claim 14 wherein said means for attaching said strip to the athletic uniform comprises at least two interior apertures disposed through said first surface and said second surface, wherein said apertures are dimensioned and positioned to allow a shoelace to be inserted through both of said apertures.

17. The athletic uniform of claim 10 wherein said means for attaching said strip to the athletic uniform is a male snap portion dimensioned to attach to a mating female snap portion disposed upon the athletic uniform.

18. The athletic uniform of claim 11 wherein said article of athletic clothing is selected from the group consisting of shorts, a shirt, a jersey, pants, and shoes.

19. A method for removably attaching an apparatus for releasably securing an athletic mouth guard during periods of athletic inactivity to an article of athletic clothing, said method comprising:
affixing uniform fastener means to said article of athletic clothing; and
attaching strip fastener means to an elongate strip, said strip having a hook and loop fastener portion, wherein said strip is sufficiently flexible to wrap about the athletic mouth guard and said strip is dimensioned, by both length and width, to secure the athletic mouth guard to said article of athletic clothing, to said uniform fastener means;
attaching said strip fastener means to said uniform;
holding said athletic mouth guard proximate to said strip; and
wrapping said strip about said athletic mouth guard such that said first surface engages said second surface to effectively secure said athletic mouth guard.
